(19) United States
(12) Patent Application Publication
(10) Pub. No.: US 2007/0093703 A1
(43) Pub. Date: Apr. 26, 2007

(54) System and method for non-endoscopic optical biopsy detection of diseased tissue

(76) Inventors: Chester E. Sievert JR., Mahtomedi, MN (US); Scott Wilson, Maple Grove, MN (US); Ron Zimmerman, Woodbury, MN (US); Jim Pokorney, Northfield, MN (US)

Correspondence Address: FISH & RICHARDSON, PC, P.O. BOX 1022, MINNEAPOLIS, MN, 55440-1022, US

(21) Appl. No.: 11/585,785

(22) Filed: Oct. 23, 2006

Related U.S. Application Data
(60) Provisional application No. 60/730,209, filed on Oct. 24, 2005.

(51) Int. Cl. A61B 5/00

(52) U.S. Cl. 600/343; 600/317; 600/342

(57) ABSTRACT

A catheter has an elongated catheter shaft adapted for introduction into a body passageway of a patient. At least one optical fiber extends through the catheter shaft. The optical fiber has a distal end positioned at or near a distal end of the catheter for illuminating tissue and receiving light energy from tissue at the location of the distal end of the tip. A distal region of the catheter includes a deformed portion having a crest offset from a longitudinal axis of the catheter shaft. A distal tip of the optical fiber is positioned at the crest to increases the likelihood of the distal tip contacting tissue of a wall of the body passageway.

### REFERENCE TO PRIORITY DOCUMENT

This application claims priority of co-pending U.S. Provisional Patent Application Ser. No. 60/730,209, filed Oct. 24, 2005. Priority of the aforementioned filing date is hereby claimed and the disclosure of the Provisional Patent Application is hereby incorporated by reference in its entirety.

### BACKGROUND

The present application relates to a system for diagnosing and performing interventional procedures within body cavities or passageways using an insertable catheter.

The field of minimally invasive surgery is experiencing dramatic growth. Laparoscopic appendectomy, cholecystectomy and various gynecological procedures have become widely adopted in clinical practice. When performed safely, the minimally invasive alternatives to traditional surgical intervention can reduce costs of care by shortening hospital stays and recuperation times. They also provide additional benefits such as reduced patient discomfort and better cosmetic results.

In minimally invasive surgery, a portal is formed in the patient's skin and tools such as catheters are inserted into the body cavity. Alternately, the tool is inserted into a pre-existing entryway of the body, such as the mouth or nose. Numerous type of catheter devices have been developed for minimally invasive medical diagnosis and treatment of various conditions. Such devices are designed for sampling tissue within the body, for example in endoscopic, laparoscopic and vascular procedures to analyze tissue and/or retrieve biopsy samples for analysis and identification of tissue types.

The catheter typically includes an analysis means, such as an optical fiber that extends through the device. The catheter can also include tools, such as biopsy forceps, that generally include small cutting jaws at the distal end, operated remotely from the proximal end after the distal end of the device has been positioned or navigated to the site of interest. The optical fiber may be connected at a proximal end to electro-optical spectral analysis equipment. The distal tip of the fiber is adapted to illuminate and receiving light energy from tissue at the location of the tip. In this regard, it is desirable for the distal tip of the optical fiber to be adjacent to or in contact with the tissue to be analyzed.

It can be difficult for the operator to position the distal tip of the fiber optic adjacent the wall of a body passageway. One reason for this is because the fiber optic is aligned with the central axis of the catheter and, therefore, is offset from the wall of the body passageway. It would be desirable for the optical fiber to be positioned in a manner that increases the likelihood of the fiber contacting the relevant body tissue.

### SUMMARY

Disclosed is a system used to spectrophotometrically characterize tissue inside a body cavity or body passageway without the use of an endoscope. The system generally includes a software controlled spectrophotometer, a diagnostic module, and a fiber-optic probe or catheter. The catheter is adapted to increases the likelihood that a detection region of the catheter will contact tissue when the catheter is positioned inside a body passageway. The system is adapted to make continuous measurements with a fiber optic probe or catheter while the catheter is in motion.

In on aspect, there is disclosed a catheter, comprising an elongated catheter shaft adapted for introduction into a body passageway of a patient; at least one optical fiber extending through the catheter shaft, the optical fiber having a proximal end coupled to electro-optical spectral analysis equipment and a distal end positioned at or near a distal end of the catheter for illuminating tissue and receiving light energy from tissue at the location of the distal end of the tip; wherein a distal region of the catheter includes a deformed portion having a crest offset from a longitudinal axis of the catheter shaft and wherein a distal tip of the optical fiber is positioned at the crest to increases the likelihood of the distal tip contacting tissue of a wall of the body passageway.

In another aspect, there is disclosed a method of using a catheter comprising: providing a catheter having at least one optical fiber extending through a catheter shaft wherein a distal region of the catheter includes a deformed portion having a crest offset from a longitudinal axis of the catheter shaft and wherein a distal tip of the optical fiber is positioned at the crest to increases the likelihood of the distal tip contacting tissue of a wall of the body passageway; inserting the catheter into a body passageway of a patient; and manipulating the catheter such that the crest is adjacent tissue to be examined.

Other features and advantages should be apparent from the following description of various embodiments, which illustrate, by way of example, the principles of the disclosed devices and methods.

### BRIEF DESCRIPTION OF THE DRAWINGS

FIG. 1 shows a schematic view of a detection system that can be used to spectrophotometrically characterize tissue inside a body without the use of a remote vision system, such as an endoscope.

FIGS. 2A-2C show a first embodiment of a catheter that includes a pre-shaped distal region.

FIGS. 3A-3C show another embodiment of the catheter with an angled distal region.

FIGS. 4, 5A and 5B show another embodiment of the catheter wherein the distal region is of a preformed shape.

FIG. 6 shows yet another embodiment of the catheter, which includes an inflatable balloon at the distal region.

FIG. 7 shows yet another embodiment of the catheter wherein a wire structure is movably positioned at the distal region of the catheter.

### DETAILED DESCRIPTION

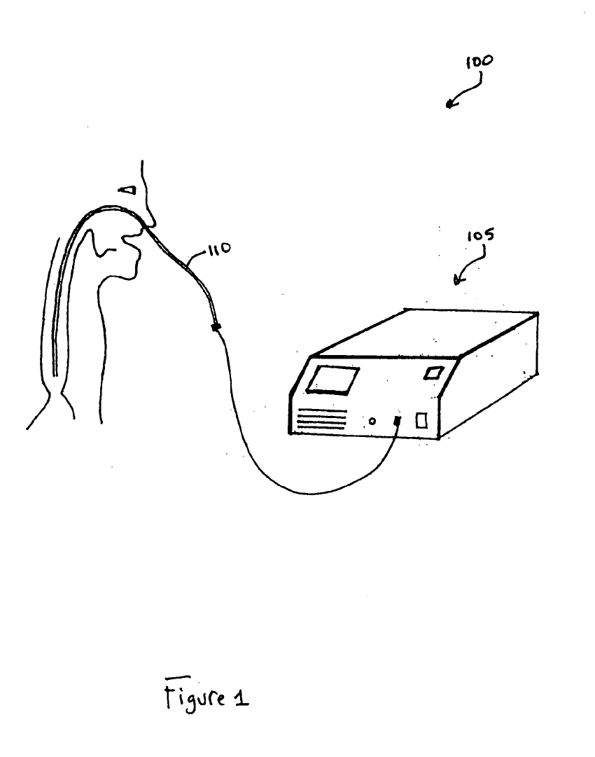

FIG. 1 shows a schematic view of a detection system 100 that can be used to spectrophotometrically characterize tissue inside a body without the use of a remote vision system, such as an endoscope. The system 100 generally includes a spectrophotometer/diagnostic module or electro-optical spectral analysis equipment 105, which is connected to a catheter 110 that includes a fiber-optic probe. The catheter 110 is configured for introduction into the body of a patient and navigation into a location of interest, such as into the esophagus via the patient's mouth. It should be appreciated that the catheter 110 can be introduced into the body in different manners and different locations.

The system 100 may be used with any type of electro-optical technique for intrinsic and/or extrinsic applications. This may include systems which use viewing or imaging, systems which use illumination with white light or specific wavelength(s) light to excite native tissue and/or dyes in the area of interest, and spectroscopic techniques to identify tissue types by spectral analysis of light returned from tissue illuminated with said light. Such spectroscopic techniques utilize the property of certain tissue types to absorb, reflect, or fluoresce light having characteristic wavelengths.

An optical fiber(s) extends through the catheter 110. At a proximal end, the catheter 110 and the optical fiber are connected to the electro-optical spectral analysis equipment 105. A distal tip of the optical fiber is positioned at or near a distal end of the catheter 110 for illuminating tissue and receiving light energy from tissue at the location of the distal end of the tip.

The distal region of the catheter 110 can optionally be equipped with one or more interventional devices or tools, such as cutting jaws, brushes, scalpel, suction device(s) or others that are controlled by electro or mechanical means through the catheter body to a control handle at the proximal end of the catheter 110. If such tools are present, the optical fiber may be positioned co-axially or external to the mechanics of the tool at a zone of contact such that in the case of jaws tool, a biopsy sample can be taken at the spot of measurement. The tool(s) can be adapted for use internally of the body, for example in connection with endoscopic, laparoscopic or vascular procedures. The tools are adapted for engaging tissue diagnosed by the spectroscopic diagnosis system in order to perform an interventional procedure on the tissue. The catheter can include a control handle portion at a proximal region of the catheter, a middle portion which extends over the main length of the catheter, and a distal end which includes tools (such as opposed forceps or cutting jaws) distal of the optical fiber.

The catheter 110 can be made from a flexible polymeric material, such as, for example, polyurethane, PTFE, PVC, etc., or any other suitable material that permits passage into a body cavity or passageway of a patient. The material can be a lubricous material that facilitates passage into the body. The size of the catheter 110 can be selected to conform to or otherwise compliment the size and location of the body cavity of interest. The catheter 110 is capable of maneuvering through the body with sufficient columnar stiffness, shaft torqueability, and distal tip flexibility. The distal section of the catheter may be capable of constant tissue contact with the target area, such as esophagus, to ensure spectroscopic light is collected through fiber optics.

The analysis equipment 105 includes hardware and/or software that permits continuous or intermittent, scanning of long segments of tissue. A tissue recognition method is configured to render spectral analysis of tissue and may permit a user to determine or document where the catheter 110 is located in the patient's anatomy either electronically or by physical measurement.

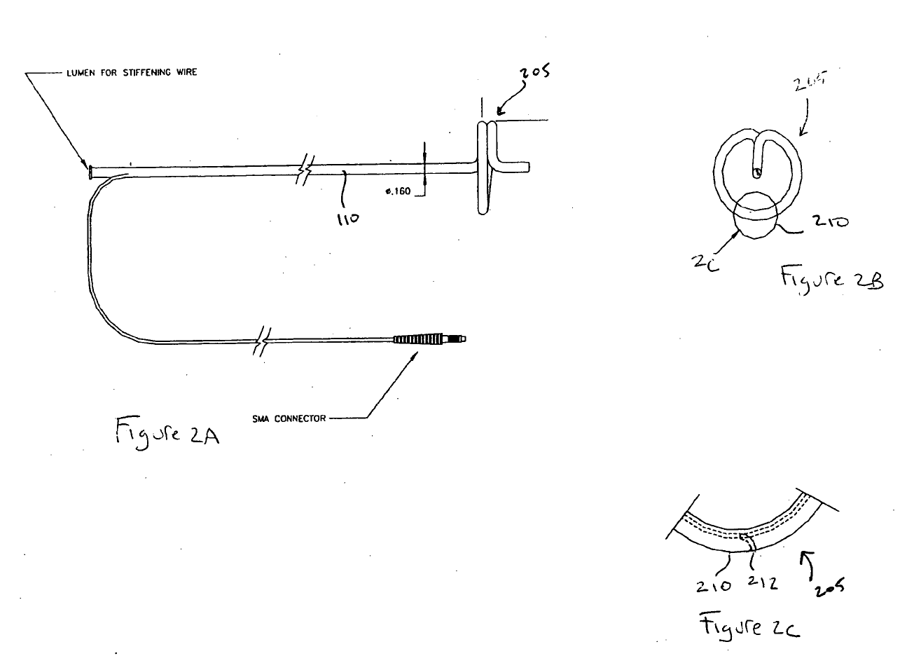

FIGS. 2-8 shows various embodiments of a catheter 110 that is used in the system 100. FIGS. 2A-2C shows a first embodiment of a catheter 110 that includes a deflectable distal region or distal tip 205. The catheter 110 includes a mechanism that enables the distal region 205 of the catheter 110 to assume a deformed (i.e., a non-straight) or otherwise predetermined shape, such as an angled or curved shape, from a previously straight or non-deformed shape. FIG. 2A shows the catheter 110 with a curved shape at the distal region 205. As best shown in the side view of FIG. 2A and the front view of FIG. 2B, the distal region 205 is generally round or follows an annular path such that a crest 210 is formed on the catheter 110.

Alternately, the distal region can have a non-curved shape, such as a triangular shape, that provides a crest. The crest 210, which is shown in an enlarged view in FIG. 2C, is offset a predetermined radius from the longitudinal axis of the catheter when the catheter is generally straightened. The offset positioning of the crest 210 increases the likelihood that the crest region (and therefore the optical fiber) of the catheter will contact tissue when the catheter is positioned inside a body passageway. As shown in FIG. 2C, the catheter includes an optical fiber, or other detection means, that has a distal tip 212 that is positioned at or near the crest 210. The distal tip of the optical fiber is positioned in a manner that increases the likelihood of the distal tip contacting tissue. Thus, the distal tip of the optical fiber is offset from the longitudinal axis of the catheter shaft.

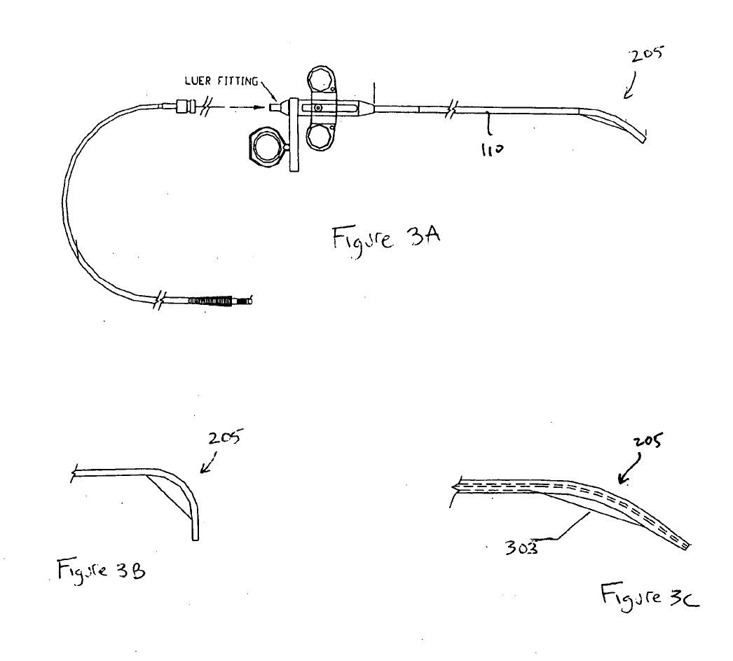

FIGS. 3A-3C shows another embodiment of the catheter 110 with an angled distal region 205. The distal region 205 has a preformed shape that angles off from a straight axis. This embodiment of the catheter 110 includes a mechanism for deflecting the distal tip or region of the catheter such that the distal tip assumes a new shape adapted to maximize contact with tissue when the catheter is in a body passageway. For example, the catheter 110 can include a pull wire 303 (or other type of actuator) that can be actuated to deflect the catheter to cause the deformed region to transition from a first shape (such as straight) to the deformed shape. FIG. 3B shows the distal region 210 in a contracted state such that the distal region is at an angle of approximately ninety degrees from another portion of the catheter. As shown in FIG. 3B, the distal region 210 can be relaxed such that the distal region is aligned at a lesser angle with respect to the remainder of the catheter. In this embodiment, the crest is at the distal end of the catheter shaft.

In an exemplary embodiment, the deflection mechanism may comprise one or more pull wires that extend through the catheter 110. The pull wires, if used, are connected to an actuator, such as sliding handle, at the proximal end of the catheter 110. The actuator is actuated to cause the pull wires to move axially, which causes the distal region 205 to assume the predetermined shape.

When the distal region 205 is angled (as in FIGS. 3A-3C), the distal tip of the optical fiber is located at the distal tip of the catheter 110. When the distal region has a curved shape (as in FIGS. 2A-2C), the distal tip of the optical fiber is located at crest of the curve or at an outside wall of the curved shape.

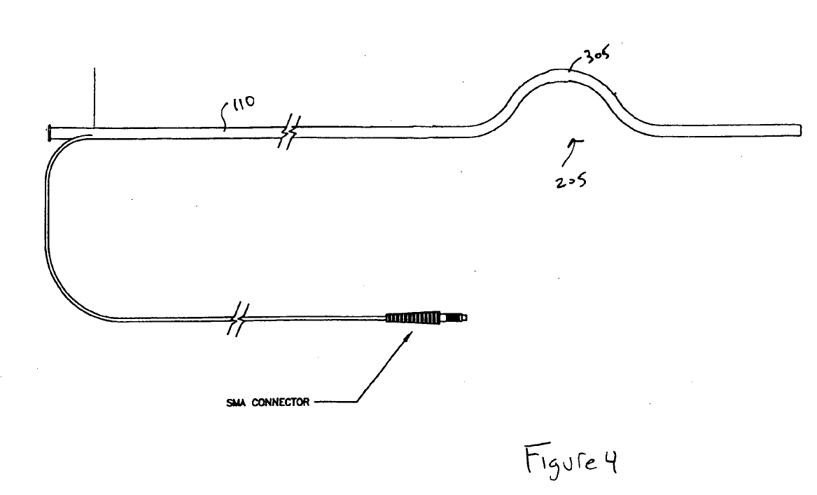

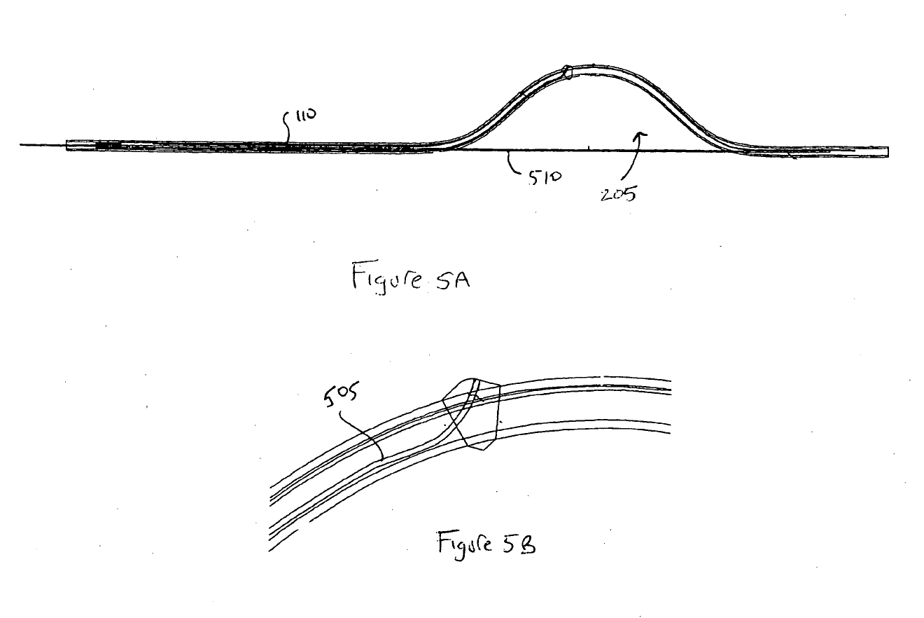

FIGS. 4, 5A, and 5B show another embodiment of the catheter 110 wherein the distal region 205 is of a preformed shape. The pre-shape can vary although the shape is selected such that it encourages contact between at least a portion of the catheter and the tissue of the body cavity in which the catheter is positioned. For example, the distal region 205 can have a “J” or “C” shaped distal region. That is, the distal region can be shaped so that it curves or bends away from the longitudinal axis of the catheter and then curves back toward the axis so as to provide a curved shape to a region of the catheter. In this manner, the catheter includes a “hump” shape along at least a portion of its length. Alternately, the distal region can be “J” shaped such that it bends away from the longitudinal axis with the distal tip of the catheter shaft offset from the longitudinal axis. As in the previous embodiments, the crest of the hump or a region near the crest can includes a distal tip of a fiber optic or other type of detector. FIG. 5B shows an enlarged view of the “hump” region of the catheter and shows how the distal tip of a fiber optic cable 505 is positioned at or near the crest. Other applications may not require physical contact with the tissue and catheter.

The distal region 205 in the embodiment of FIGS. 4 and 5 is “C” shaped with the “C” having a crest 305 that is offset from the longitudinal axis of the catheter 110. The offset crest 305 is positioned such that it will likely contact or otherwise rest against the wall of a body cavity in which the catheter 110 is positioned. As mentioned, the distal tip of the optical fiber protrudes out of the catheter 110 at the location of the crest 305. In this manner, the optical fiber contacts the tissue of the body cavity. As in the previous embodiment, the catheter can include a deflection mechanism, such as a pull wire 510 (FIG. 5) that can be used to deflect or otherwise deform the shape of the catheter.

The catheter 110 is sufficiently flexible such that it can be at least temporarily straightened when positioned into straight catheter or when a stiff, straight catheter is positioned inside the catheter 110. However, when the catheter 110 is in its default state, the catheter 110 has the preshape in its distal region 205.

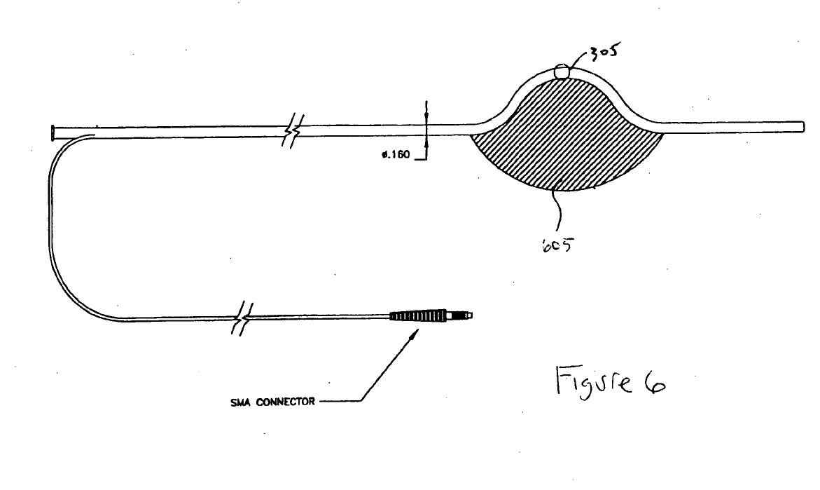

FIG. 6 shows yet another embodiment of the catheter 110, which includes an inflatable balloon 605 at the distal region 205. Like the embodiment shown in FIGS. 3 and 4, the embodiment of FIG. 6 includes a preshaped distal region 205, such as in the shape of a “C”. It should be appreciated that the preshaped region can have various other shapes for any of the embodiments described herein. The optical fiber distal tip is positioned at the crest 305 of the pre-shaped configuration in the distal region 205. The balloon 605 extends radially outward from the preshaped distal region opposite the direction of the offset preshaped region. When the balloon is inflated, the expansion of the balloon causes the crest 305 to press against the wall of the body cavity thereby encouraging contact between the distal tip of the optical fiber and the tissue on the wall.

In an embodiment, the optical fibers are also positioned around the outer wall of the balloon 510. Thus, inflation of the balloon 510 moves the fibers radially outward to encourage contact between the fibers and the desired tissue.

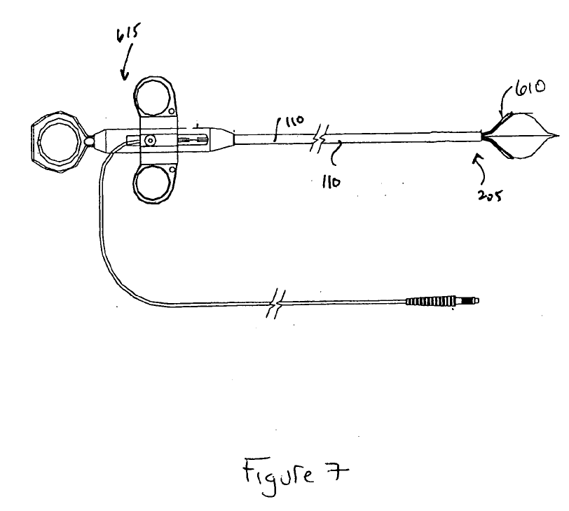

FIG. 7 shows yet another embodiment of the catheter 110 wherein a wire structure 610 is movably positioned at the distal region 205 of the catheter 110. The wire structure 610 is biased toward an expanded state shown in FIG. 6. When in the expanded state, the wire structure 610 has distal tips or some other portion that are radially offset from the axis of the catheter. The tip of optical fiber is positioned at the radially-offset portion such that the wire structure 610 positions the optical fiber in contact with tissue. There may be more than one and as many as several optical fibers in the catheter making several positions radially around the body passageway, such as the esophagus. It should be appreciated that the body passageway can vary and can include, for example, the colon, cervix, lung, urethra, etc. In this case an optical switch or scanner could be incorporated in the device to sequentially make measurements.

The wire structure 610 is configured to be retracted or withdrawn into the catheter body 110 or into a sleeve coupled to the catheter body such as by manipulating an actuator 615 at the proximal end of the catheter. When withdrawn into the catheter 110, the wire structure 610 is constricted by the walls of the catheter 110 so that the structure 610 assumes a smaller size to facilitate passage of the catheter 110 into the body.

In use, the catheter 110 is introduced into a patient's body and to a target location in the body. The catheter 110 is manipulated such that the distal region 205 is in contact with tissue to be examined. The tissue is then optically scanned either at a single point or in a semi-continuous, or continuous mode by moving the catheter distal region 205 along a segment of the tissue. Advantageously, the catheter 110 is shaped such that the likelihood of continuous contact between the optical fiber and the tissue is maximized. For example, the pre-shaped distal region 205 of the embodiment shown in FIGS. 5 and 6 has an offset crest 305 that will contact the circumference of the wall of a body lumen, for example, as the catheter 110 is rotated within the body lumen.

The optical scan includes excitation of the tissue with electro-optical energy of one or more wavelengths and reading of the auto-fluorescence spectra emitted from the exited tissue. The diagnostic algorithm determines if the tissue is normal or diseased. A result could then be displayed on a console.

The optical fiber catheter 110 delivers the excitation light to the tissue and return the collected spectral signal to the equipment 105 for analysis and tissue diagnosis. The catheter 110 can make continuous tissue contact and ongoing collection of spectral signals while the catheter is being pushed, pulled and directed throughout any of the internal structures of the body. Other sources of energy, such as ultrasound, could be utilized in place of optical techniques.

The spectrophotometry equipment 105 is capable of semi-continually, or continually collecting spectral (or acoustic for instance) signal information and making a diagnosis while the catheter scans or moves throughout the body. The equipment 105 produces the excitation light (or acoustic) delivered to the tissue, collects and analyzes the returning spectral signal, make and displays the tissue diagnosis, and optionally documents the anatomical location of the catheters' distal tip.

In one embodiment, the patient is unsedated with exception to oral or nasal delivery, such as where short acting topical anesthesia is routine and standard for prevention of gagging during similar non-endoscopic catheter based procedures. The optical (or acoustic) catheter 110 is introduced until the equipment's console displays, or physical distance measurements marked on the catheter an appropriate depth of insertion. The excitation light source (or acoustic) is initiated at the time of insertion. Continuous spectral measurements are made on the way in and during removal of the catheter 110. During the procedure, anatomical location of the catheters' distal tip will be displayed on the systems' console or can be physically read from marks on the catheter. After removal of the catheter 110 from the body cavity and collection of the spectral signals, a tissue diagnosis is displayed on the systems' console.

The system 100 can be used in conjunction with other devices, for example, phototherapy devices to treat suspected or diseased tissue or with balloon catheters that are used to re-shape the body cavity in which the catheter 110 is positioned. Another example would be its use in conjunction with an RF therapy device where the catheter provides the diagnosis and the RF generator the therapy.

Although embodiments of various methods and devices are described herein in detail with reference to certain versions, it should be appreciated that other versions, embodiments, methods of use, and combinations thereof are also possible. Therefore the spirit and scope of the disclosure should not be limited to the description of the embodiments contained herein.

What is claimed:

1. A catheter, comprising:
an elongated catheter shaft adapted for introduction into a body passageway of a patient;
at least one optical fiber extending through the catheter shaft, the optical fiber having a proximal end coupled to electro-optical spectral analysis equipment and a distal end positioned at or near a distal end of the catheter for illuminating tissue and receiving light energy from tissue at the location of the distal end of the tip; and
wherein a distal region of the catheter includes a deformed portion having a crest offset from a longitudinal axis of the catheter shaft and wherein a distal tip of the optical fiber is positioned at the crest to increases the likelihood of the distal tip contacting tissue of a wall of the body passageway.

2. A catheter as in claim 1, wherein the deformed portion is curved.

3. A catheter as in claim 1, wherein the deformed portion is c-shaped.

4. A catheter as in claim 1, wherein the deformed portion follows an annular path.

5. A catheter as in claim 1, wherein the deformed portion is bent.

6. A catheter as in claim 1, further comprising a pull wire coupled to the deformed portion, wherein the deformed portion is straight in a default state and wherein the deformed portion assumes a deformed shape upon actuation of the pull wire.

7. A catheter as in claim 1, wherein the catheter shaft is sufficiently flexible such that the catheter shaft can be at least temporarily straightened when positioned into a straight catheter or when a stiff, straight catheter is positioned inside the catheter shaft.

8. A catheter as in claim 1, further comprising an inflatable balloon that extends radially-outward from the deformed portion opposite the direction of the crest.

9. A catheter as in claim 8, wherein the balloon inflates to causes the crest to press against the wall of the body cavity thereby encouraging contact between the distal tip of the optical fiber and the tissue on the wall.

10. A catheter as in claim 1, wherein at least one optical fiber is positioned around an outer wall of the balloon such that inflation of the balloon moves the optical fibers radially-outward to encourage contact between the optical fiber and the tissue.

11. A catheter as in claim 1, wherein the deformed portion comprises a wire structure movably positioned at the distal region of the catheter, the wire structure biased toward an expanded state wherein distal tips are radially offset from the longitudinal axis of the catheter and wherein the distal tip of optical fiber is positioned at the radially-offset portion of the wire structure such that the wire structure

12. A catheter as in claim 11, wherein the wire structure is configured to be retracted or withdrawn into the catheter shaft so that the wire structure assumes a smaller radial size to facilitate passage of the catheter shaft into the body passageway.

13. A catheter as in claim 1, further comprising an interventional device located at the distal end of the catheter shaft for engaging tissue diagnosed by the spectroscopic diagnosis system in order to perform an interventional procedure on the tissue.

14. A catheter as in claim 13, wherein the interventional device comprises forceps.

15. A catheter as in claim 1, further comprising a control handle portion at a proximal region of the catheter.

16. A method of using a catheter comprising:
providing a catheter having at least one optical fiber extending through a catheter shaft wherein a distal region of the catheter includes a deformed portion having a crest offset from a longitudinal axis of the catheter shaft and wherein a distal tip of the optical fiber is positioned at the crest to increases the likelihood of the distal tip contacting tissue of a wall of the body passageway;
inserting the catheter into a body passageway of a patient; and
manipulating the catheter such that the crest is adjacent tissue to be examined.

17. A method as in claim 16, further comprising using the optical fiber to optically scan the tissue by moving the crest along a segment of the tissue.

18. A method as in claim 17, wherein the optical scan includes excitation of the tissue with electro-optical energy of one or more wavelengths and reading of the auto-fluorescence spectra emitted from the exited tissue.

19. A method as in claim 18, further comprising delivering excitation light to the tissue to obtain a spectral signal; and
returning the collected spectral signal to spectrophotometry equipment for analysis and tissue diagnosis.
